(19) United States
(12) Patent Application Publication
(10) Pub. No.: US 2003/0138785 A1
(43) Pub. Date: Jul. 24, 2003

(54) In vivo protein screen based on enzyme-assisted chemically induced dimerization ("CID")

(76) Inventors: Stephan Kopytek, New York, NY (US); Virginia Cornish, New York, NY (US)

Correspondence Address: Cooper & Dunham LLP, 1185 Avenue of the Americas, New York, NY, 10036, US

(21) Appl. No.: 10/084,388

(22) Filed: Feb. 25, 2002

Related U.S. Application Data
(60) Provisional application No. 60/343,467, filed on Dec. 21, 2001.

(51) Int. Cl. C12Q 1/68; G01N 33/53; C12N 15/85

(52) U.S. Cl. 435/6; 435/7.1; 435/455

(57) ABSTRACT

A method for identifying which protein from a pool of candidate proteins catalyzes in a cell a bond forming reaction between a first substrate and a second substrate, comprising:

(a) providing a dimeric small molecule which comprises a known moiety that binds a known receptor domain covalently linked with a moiety that contains the first substrate;

(b) introducing the dimeric molecule into a cell which comprises

i) a first fusion protein comprising the known receptor domain,

ii) a second fusion protein comprising the second substrate,

iii) a protein from the pool of candidate proteins, and

iv) a reporter gene wherein expression of the reporter gene is conditioned on the proximity of the first fusion protein to the second fusion protein;

(c) permitting the dimeric molecule to bind to the first fusion protein and to enzymatically form a bond with the second fusion protein so as to activate the expression of the reporter gene;

(d) selecting which cell expresses the reporter gene; and

(e) identifying the protein that catalyzes the bond formation reaction in the cell between the first substrate and the second substrate. The method is also adapted to identify which substrate from a pool of candidate substrates is selected in a cell by a known enzyme for a bond forming reaction between the substrate and a known amino acid. Also, cells, compounds and kits for carrying out the methods.

This application claims the benefit of U.S. Provisional Application No. 60/343,467, filed Dec. 21, 2001, the contents of which are hereby incorporated by reference.

Throughout this application, various publications are referenced by Arabic numerals in parentheses. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein.

### FIELD OF INVENTION

This invention relates to the field of screening a group of target proteins or chemicals using techniques of chemically induced dimerization (“CID”).

### BACKGROUND OF THE INVENTION

Several in vivo screens exist based on protein-protein interaction. A yeast genetic screening method, known as the Yeast Two-Hybrid system, has been developed for specifically identifying protein-protein interactions in an in vivo system (1a). The yeast Two-Hybrid system relies on the interaction of two fusion proteins to bring about the transcriptional activation of a reporter gene such as E.coli derived β-galactosidase (Lac Z). One fusion protein comprises a preselected protein fused to the DNA binding domain of a known transcription factor. The second fusion protein comprises a polypeptide from a cDNA library fused to a transcriptional activation domain. In order for the reporter gene to be activated, the polypeptide from the cDNA library must bind directly to the preselected target protein. Yeast cells harboring an activated reporter gene can be differentiated from other cells and the cDNA encoding for the interacting polypeptides can be easily isolated and sequenced. However, this assay is unsuited for screening small molecule-protein interactions because it relies solely on genetically encoded fusion protein interaction.

The subsequently developed Yeast Three-Hybrid system is able to screen for a small molecule-protein interaction (1b). This system is based on the principle that small ligand-receptor interactions underlie many fundamental processes in biology and form the basis for pharmacological intervention of human diseases in medicine. This system is adapted from the yeast two-hybrid system by adding a third synthetic hybrid ligand. The feasibility of this system was demonstrated using as the hybrid ligand a dimer of covalently linked dexamethasone and FK506. The system used yeast expressing fusion proteins consisting of a) hormone binding domain of the rat glucocorticoid receptor fused to the LexA DNA-binding domain and b) FKBP12 fused to a transcriptional activation domain. When the yeast was plated on medium containing the dexamethasone-FK506 heterodimer, the reporter genes were activated. The reporter gene activation is completely abrogated in a competitive manner by the presence of excess FK506. Using this system, a screen was performed of a Jurkat cDNA library fused to the transcriptional activation domain in yeast in the presence of a dexamethasone-FK506 heterodimer. The yeast in this system expressed the hormone binding domain of rat glucocorticoid receptor/DNA binding domain fusion protein. Overlapping clones of human FKBP12 were isolated. The three-hybrid system can be used to discover receptors for small ligands and to screen for new ligands to known receptors.

Further improvements led to a chemically induced dimerization (“CID”) system that uses small molecule induced protein dimerization to screen for catalysis in vivo. WO 01/53355 describes a number of screening approaches using this system, which is refered to as the basic CID system, including the use of small molecules to induce protein dimerization to screen cDNA libraries based on binding, or small molecules with cleavable linkers to screen cDNA libraries based on catalysis. The contents of WO 01/53355 are hereby incorporated by reference. The CID technology offers a promising approach to screening cDNA libraries based on function because a variety of activities can be assayed simply by changing one of the CID ligand/receptor pairs or by changing the bond between the CID ligands. In the basic CID system, the dimerizer molecule induces dimerization of the two halves of a reporter protein since each domain of the reporter protein is fused to a receptor for one of the two linked ligands (1, 2). The resultant ternary complex can be detected in vitro by gel filtration analysis (2); in vivo by the yeast three-hybrid (Y3H) system (1). The basic CID system is show in FIG. 1.

The basic CID approaches rely on 4 non-covalent interactions existing simultaneously for the reporter protein to be activated. Specifically, 1) the DNA-binding protein-DNA interaction, 2) the 1^{st} ligand-receptor interaction, 3) the 2^{nd} ligand-receptor interaction, and 4) the activation domain-transcription machinery interaction. This is useful in certain types of screens.

However, another desirable screen is for enzymes that can form covalent bonds between two proteins or a small non-peptide molecule and a protein. Referring to the four interactions of the basic CID system, a desirable screen would have an enzyme form a covalent bond instead of the non-covalent interaction 2 or 3. Such a screen is provided by this invention.

### SUMMARY OF THE INVENTION

This invention provides a method for identifying which protein from a pool of candidate proteins catalyzes in a cell a bond forming reaction between a first substrate and a second substrate, comprising:

(a) providing a dimeric small molecule which comprises a known moiety that binds a known receptor domain covalently linked with a moiety that contains the first substrate;

(b) introducing the dimeric molecule into a cell which comprises

i) a first fusion protein comprising the known receptor domain,

ii) a second fusion protein comprising the second substrate,

iii) a protein from the pool of candidate proteins, and

iv) a reporter gene wherein expression of the reporter gene is conditioned on the proximity of the first fusion protein to the second fusion protein;

(c) permitting the dimeric molecule to bind to the first fusion protein and to enzymatically form a bond with the second fusion protein so as to activate the expression of the reporter gene;

(d) selecting which cell expresses the reporter gene; and

(e) identifying the protein that catalyzes the bond formation reaction in the cell between the first substrate and the second substrate.

The method is readily adapted to identify which substrate from a pool of candidate substrates is selected in a cell by a known enzyme for a bond forming reaction between the substrate and a known amino acid.

Also provided by this invention is a transgenic cell comprising

(a) a dimeric small molecule which comprises a moiety known to bind a receptor domain covalently linked to a first substrate of an enzyme;

(b) nucleotide sequences which upon transcription encode

i) the enzyme,

ii) a first fusion protein comprising the receptor domain, and

ii) a second fusion protein comprising a second substrate of the enzyme; and

(c) a reporter gene wherein expression of the reporter gene is conditioned on the proximity of the first fusion protein to the second fusion protein.

The invention also provides a kit for detecting bond formation by an enzyme between a first substrate and a second substrate in a cell, comprising

(a) a host cell containing a reporter gene that is expressed only when bound to a DNA-binding domain and when in the proximity of a transcription activation domain;

(b) a first vector containing a promoter that functions in the host cell and a DNA encoding a DNA-binding domain;

(c) a second vector containing a promoter that functions in the host cell and a DNA encoding a transcription activation domain;

(d) a third vector containing a promoter that functions in the host cell;

(e) a dimeric small molecule which comprises a moiety known to bind a receptor domain and a moiety containing the first substrate of the enzyme;

(f) a means for inserting into the first vector or the second vector a DNA encoding a receptor domain in such a manner that the receptor domain and the DNA-binding domain are expressed as a fusion protein;

(g) a means for inserting into the first vector or the second vector a DNA encoding a protein containing the second substrate of the enzyme in such a manner that the protein and the transcription activation domain are expressed as a fusion protein;

(h) a means for inserting into the third vector a DNA encoding the enzyme; and

(h) a means for transfecting the host cell with the first vector, the second vector, and the third vector,

wherein bond formation by the enzyme between the first substrate and the second substrate results in a measurably greater expression of the reporter gene then in the absence of bond formation by the enzyme.

The invention also provides a small molecule compound having the structure:

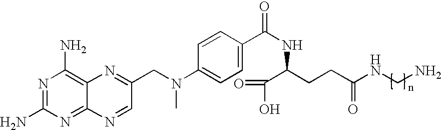

wherein n is an integer from 1 to 20; or, in other embodiments, n can be from 2 to 12; or n can be from 3 to 9; or n is 5.

### DESCRIPTION OF THE FIGURES

FIG. 1. The basic CID system. Presence of the dimeric small molecule dimerizes the two fusion proteins. One fusion protein comprises a DNA-binding domain fused to a receptor domain; and a second fusion protein comprises a transcription activation domain fused to another receptor domain. By dimerizing the two fusion proteins, the dimeric small molecule brings into proximity the DNA-binding domain and the transcription activation domain, thus activating the cellular readout.

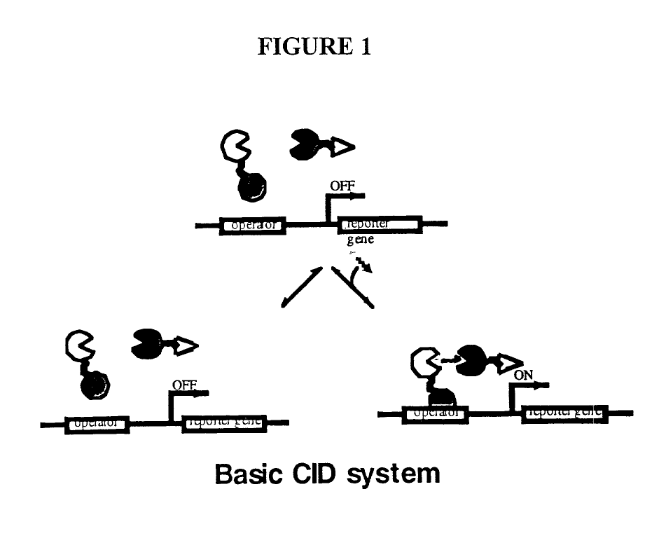

FIG. 2. The yeast three-hybrid (Y3H) system. The small molecule dexamethasone-FK506 mediates the dimerization of the LexA-GR (glucocorticoid receptor) and B42-FKBP12 protein fusions. Dimerization of the DNA-binding domain of the fusion protein LexA-GR and the activation domain of the fusion protein B42 FKB72 activates transcription of the lacZ reporter gene.

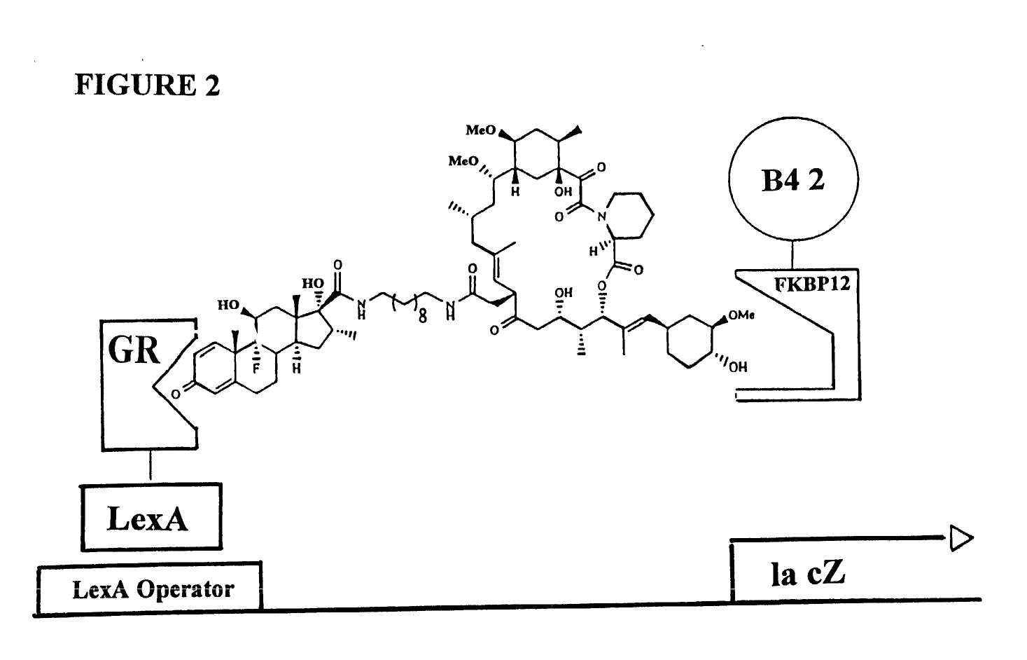

FIG. 3. The enzyme-assisted chemically induced dimerization (“eACID”) system. (1) is the reporter sequence having a reporter gene and at least one DNA binding site, which upon activation directs transcription of the gene. (2) and (3) are the fusion proteins, one of which comprises a DNA-binding domain fused to a receptor domain, and the other comprises a transcription activation domain fused to another receptor domain. However, in eACID one of the receptor domains is such that it does not spontaneously interact with the dimeric small molecule, but rather requires “assistance” of an enzyme. (4) is the dimeric small molecule consisting of two ligand halves each specific for the corresponding receptor domain. As noted, one of the ligand halves requires “assistance” of an enzyme to interact with its receptor domain. (5) is the enzyme being screened for, which “assists” the interaction between one of the ligand halves of the dimeric small molecule and one of the receptor domains.

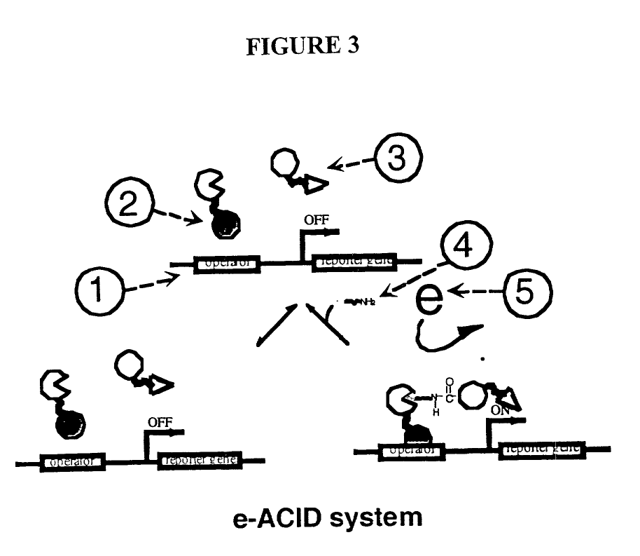

FIG. 4. Examples of known ligands: dexamethasone (A), FK506 (B), and methotrexate (C).

FIG. 5. Examples of DEX-DEX molecules with various linkers.

FIG. 6. Synthesis of the small-molecule MP5.

FIG. 7. MP5 Competition Assay. X-gal plate assay of Dexamethasone-MTX (D8M)-induced lacZ transcription and MTX-amine (MP5) inhibition of D8M-induced transcription. Yeast strains containing a lacZ reporter gene and different LexA and/or B42-chimeras were grown on X-gal indicator plates that contained different combinations of D8M, MTX, and/or MP5. Columns A through H on each plate correspond to yeast strains containing different LexA- and/or B42-chimeras: A, LexA-Sec16p, B42-Sec6p. A is a direct protein-protein interaction used as a positive control. B, LexA, B42. C, LexA-eDHFR, B42-rGR. D, LexA-mDHFR, B42-rGR. E, LexA-rGR, B42-eDHFR. F, LexA-rGR, B42-mDHFR. G, LexA-eDHFR, B42. H, LexA, B42-rGR. X-gal plates 1 through 6 have different small molecule combinations: 1, 1 μM D8M; 2, 10 μM MP5; 3, 10 μM MTX; 4, 1 μM D8M and 10 μM MTX; 5, 1 μM D8M and 10 μM MP5; 6, no small molecule.

FIG. 8. SNase expression, purification and immunodetection. Lanes 1 through 3 are coomassie stained fractions from the SNase purification; lanes 4 and 5 correspond to Western analysis of purified SNase. 1, crude yeast extract; 2, 3, 4, and 5, purified SNase.

FIG. 9. MALDI-MS of purified SNase.

FIG. 10. Examples of some Transglutaminase substrates.

FIG. 11. Examples of some Transglutaminase substrates, which are amines, for which microbial transglutaminase (“MTG”) has been shown to have specificity.

### DETAILED DESCRIPTION OF THE INVENTION

This invention provides a method for identifying which protein from a pool of candidate proteins catalyzes in a cell a bond forming reaction between a first substrate and a second substrate, comprising:

(a) providing a dimeric small molecule which comprises a known moiety that binds a known receptor domain covalently linked with a moiety that contains the first substrate;

(b) introducing the dimeric molecule into a cell which comprises

i) a first fusion protein comprising the known receptor domain,

ii) a second fusion protein comprising the second substrate,

iii) a protein from the pool of candidate proteins, and

iv) a reporter gene wherein expression of the reporter gene is conditioned on the proximity of the first fusion protein to the second fusion protein;

(c) permitting the dimeric molecule to bind to the first fusion protein and to enzymatically form a bond with the second fusion protein so as to activate the expression of the reporter gene;

(d) selecting which cell expresses the reporter gene; and

(e) identifying the protein that catalyzes the bond formation reaction in the cell between the first substrate and the second substrate.

In the method, the protein can be encoded by a DNA selected from the group consisting of genomic DNA, cDNA and synthetic DNA.

The pool of candidate proteins can be obtained by combinatorial techniques.

In the method, the steps (b)-(e) of the method can be iteratively repeated in the presence of a preparation of random proteins for competitive enzymatic bond formation so as to identify a protein having enhanced enzymatic activity.

The cell can be an insect cell, a yeast cell, a bacterial cell, or a mammalian cell. In specific embodiments, the cell can be S. cerevisae or E. coli.

The first fusion protein can further comprise a DNA binding domain, and the second fusion protein further comprise a transcription activation domain. Alternatively, the first fusion protein can further comprises a transcription activation domain, and the second fusion protein further comprise a DNA binding domain. The the DNA-binding domain can be LexA, Gal4 or VP16. The transcription activation domain can be B42.

The known moiety that binds a known receptor domain can be a Methotrexate moiety or an analog thereof. The known receptor domain can dihydrofolate reductase (“DHFR”) generally, or the E. coli DHFR (“eDHFR”). Alternatively, the pairing can be dexamethasone/glucocorticoid receptor, FK506/FKBP12, AP series of synthetic FK506 analogs/FKBPs, tetracycline/tetracycline repressor, cephem/penicillin binding protein. The penicillin binding domain can be from Streptomyces R61.

The first fusion protein can be eDHFR-LexA or R61-LexA. Alternatively, the first fusion protein can be eDHFR-B42 or R61-B42.

The reporter gene can be Lac Z, ura 3, GFP, β-lactamase, luciferase or an antibody coding region. In one embodimet it is Lac Z.

The first substrate can be an amine. Alternatively, the second substrate can be an amine. Generally, the system can be constructed to correspond to the enzyme specificity and/or to account for endogenous celullar proteins.

In certain embodiments, the second substrate is an amino acid sequence containing a lysine; is an amino acid sequence containing a glutamine; is an amino acid sequence containing -leucine-glycine-glutamine-glycine-; is an amino acid sequence containing -leucine-glutamine-glycine-glycine-; is an amino acid sequence containing -leucine-leucine-glutamine-glycine-; or is a staphylococcal nuclease (“SNase”) modified to contain an amino acid sequence containing a glutamine. Alternatively, a thioredoxin modified to containing an amino acid sequence containing a glutamine, or any other protein used as “peptamers” (28).

The protein that catalyzes bond formation can be a transglutaminase; in specific embodments it is a microbial transglutaminase, a tissue transglutaminase, or Factor XIIIA.

The dimeric small molecule can have the structure:

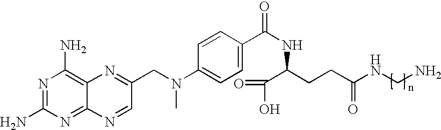

wherein n is an integer from 1 to 20; or, in other embodiments, n can be from 2 to 12; or n can be from 3 to 9; or n is 5.

Also provided by this invention is a new protein having enzymetic activity identified by the methods of this invention.

The method is readily adapted to identify which substrate from a pool of candidate substrates is selected in a cell by a known enzyme for a bond forming reaction between the substrate and a known amino acid, comprising the steps:

(a) providing a dimeric small molecule which comprises the substrate covalently linked to a moiety known to bind a receptor domain;

(b) introducing the dimeric molecule into a cell which comprises

i) a first fusion protein comprising the receptor domain,

ii) a second fusion protein comprising the known amino acid,

iii) the known enzyme, and

iv) a reporter gene wherein expression of the reporter gene is conditioned on the proximity of the first fusion protein to the second fusion protein;

(c) permitting the dimeric molecule to bind to the first fusion protein and to enzymatically form a bond with the second fusion protein so as to activate the expression of the reporter gene;

(d) selecting which cell expresses the reporter gene; and

(e) identifying the substrate selected by the known enzyme in the cell for the bond forming reaction between the substrate and the known amino acid.

The pool of candidate substrates can be obtained by combinatorial techniques.

Also, the steps (b)-(e) of the method can be iteratively repeated in the presence of a preparation of random substrates for competitive enzymatic bond formation so as to identify a substrate competitively selected by the known enzyme.

The cell, fusion proteins, reporter gene, and enzyme can be varied in the method of identifying a substrate as described above for the method of idenifying a protein that catalyzes a bond forming reaction.

Also provided by this invention is a transgenic cell comprising

(a) a dimeric small molecule which comprises a moiety known to bind a receptor domain covalently linked to a first substrate of an enzyme;

(b) nucleotide sequences which upon transcription encode

i) the enzyme,

ii) a first fusion protein comprising the receptor domain, and

ii) a second fusion protein comprising a second substrate of the enzyme; and

(c) a reporter gene wherein expression of the reporter gene is conditioned on the proximity of the first fusion protein to the second fusion protein.

The dimeric small molecule in the cell can have the structure:

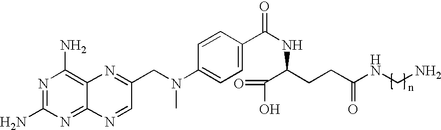

wherein n is an integer from 1 to 20; or, in other embodiments, n can be from 2 to 12; or n can be from 3 to 9; or n is 5.

The cell can be an insect cell, a yeast cell, a bacterial cell, or a mammalian cell and in a specific emboiment, a yeast cell. In specific embodiments, the cell can be S. cerevisae or E. coli.

In the cell, the first fusion protein can further comprise a DNA binding domain, and the second fusion protein further comprises a transcription activation domain. Alternatively, the first fusion protein can further comprise a transcription activation domain, and the second fusion protein further comprises a DNA binding domain. The DNA-binding domain can be LexA, Gal4 or VP16. The transcription activation domain can be B42.

In the cell, the moiety known to bind a receptor domain of the dimeric small molecule can be a Methotrexate moiety or an analog thereof; and the known receptor domain can be a dihydrofolate reductase (“DHFR”), in specific embodiments, the E.coli DHFR (“eDHFR”). Alternatively, the pairing can be dexamethasone/glucocorticoid receptor, FK506/FKBP12, AP series of synthetic FK506 analogs/FKBPs, tetracycline/tetracycline repressor, cephem/penicillin binding protein. The penicillin binding domain can be from Streptomyces R61.

The first fusion protein in the cell can be eDHFR-LexA or R61-LexA. Alternatively, the first fusion protein can be eDHFR-B42 or R61-B42.

The reporter gene in the cell can be Lac Z, ura 3, GFP, β-lactamase, luciferase or an antibody coding region; in a specific embodiment, the reporter gene is Lac Z.

The first substrate of the enzyme ca be an amine. Alternatively, the second substrate can be an amine. Generally, the system can be constructed to correspond to the enzyme specificity and/or to account for endogenous celullar proteins.

In certain embodiments, the second substrate is an amino acid sequence containing a lysine; is an amino acid sequence containing a glutamine; is an amino acid sequence containing -leucine-glycine-glutamine-glycine-; is an amino acid sequence containing -leucine-glutamine-glycine-glycine-; is an amino acid sequence containing -leucine-leucine-glutamine-glycine-; or is a staphylococcal nuclease (“SNase”) modified to contain an amino acid sequence containing a glutamine. Alternatively, a thioredoxin modified to contain an amino acid sequence containing a glutamine, or any other protein used as “peptamers” (28).

The enzyme in the cell can be a transglutaminase, in specific embodiments, the enzyme is microbial transglutaminase, a tissue transglutaminase, or Factor XIIIA.

The invention also provides a kit for detecting bond formation by an enzyme between a first substrate and a second substrate in a cell, comprising

(a) a host cell containing a reporter gene that is expressed only when bound to a DNA-binding domain and when in the proximity of a transcription activation domain;

(b) a first vector containing a promoter that functions in the host cell and a DNA encoding a DNA-binding domain;

(c) a second vector containing a promoter that functions in the host cell and a DNA encoding a transcription activation domain;

(d) a third vector containing a promoter that functions in the host cell;

(e) a dimeric small molecule which comprises a moiety known to bind a receptor domain and a moiety containing the first substrate of the enzyme;

(f) a means for inserting into the first vector or the second vector a DNA encoding a receptor domain in such a manner that the receptor domain and the DNA-binding domain are expressed as a fusion protein;

(g) a means for inserting into the first vector or the second vector a DNA encoding a protein containing the second substrate of the enzyme in such a manner that the protein and the transcription activation domain are expressed as a fusion protein;

(h) a means for inserting into the third vector a DNA encoding the enzyme; and

(h) a means for transfecting the host cell with the first vector, the second vector, and the third vector,

wherein bond formation by the enzyme between the first substrate and the second substrate results in a measurably greater expression of the reporter gene then in the absence of bond formation by the enzyme.

The elements of the kit are as described above for the methods and the cell.

The invention also provides a small molecule compound having the structure:

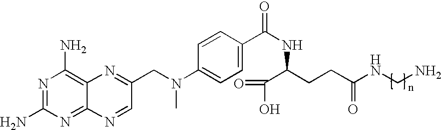

wherein n is an integer from 1 to 20; or, in other embodiments, n can be from 2 to 12; or n can be from 3 to 9; or n is 5.

The described methods, cell and kit may also be adapted to identify new protein targets for pharmaceuticals.

The described methods, cell and kit may also be adapted for determining the function of a protein, further including screening with a natural cofactor being part of the CID.

The described methods, cell and kit may also be adapted for determining the function of a protein, further including screening with a natural substrate being part of the CID.

The described methods, cell and kit may also be adapted for screening a compound for the ability to inhibit a ligand-receptor interaction.

In any of the described embodiments, each of the ligand halves of the dimeric small molecule is capable of binding to a receptor with an IC₅₀ of less than 100 nM. In a preferred embodiment, each of ligand halves of the dimeric small molecule is capable of binding to a receptor with an IC₅₀ of less than 10 nM. In the most preferred embodiment, each of the ligand halves of the dimeric small molecule is capable of binding to a receptor with an IC₅₀ of less than 1 nM.

Each of the ligand halves of the dimeric small molecule may be derived from a compound selected from the group consisting of steroids, hormones, nuclear receptor ligands, cofactors, antibiotics, sugars, enzyme inhibitors, and drugs.

Each of the ligand halves of the dimeric small molecule may also represent a compound selected from the group consisting of dexamethasone, 3,5,3′-triiodothyronine, trans-retinoic acid, biotin, coumermycin, tetracycline, lactose, methotrexate, FK506, and FK506 analogs.

In any of the described methods, the cellular readout may be gene transcription, such that change in gene transcription indicates catalysis of bond formation by the protein screened.

In the described methods, the screening is performed by Fluorescence Associated Cell Sorting (FACS), or gene transcription markers selected from the group consisting of Green Fluorescence Protein, LacZ-β-galagctosidases, luciferase, antibiotic resistant β-lactamases, and yeast markers.

The foregoing embodiments of the subject invention may be accomplished according to the guidance which follows. Certain of the foregoing embodiments are exemplified. Sufficient guidance is provided for a skilled artisan to arrive at all of the embodiments of the subject invention.

Preparation and Design of Ligand Halves of the Dimeric Small Molecule

A ligand half should bind its receptor with high affinity (≦100 nM), cross cell membranes yet be inert to modification or degradation, be available in reasonable quantities, and present a convenient side-chain for routine chemical derivatization that does not disrupt receptor binding.

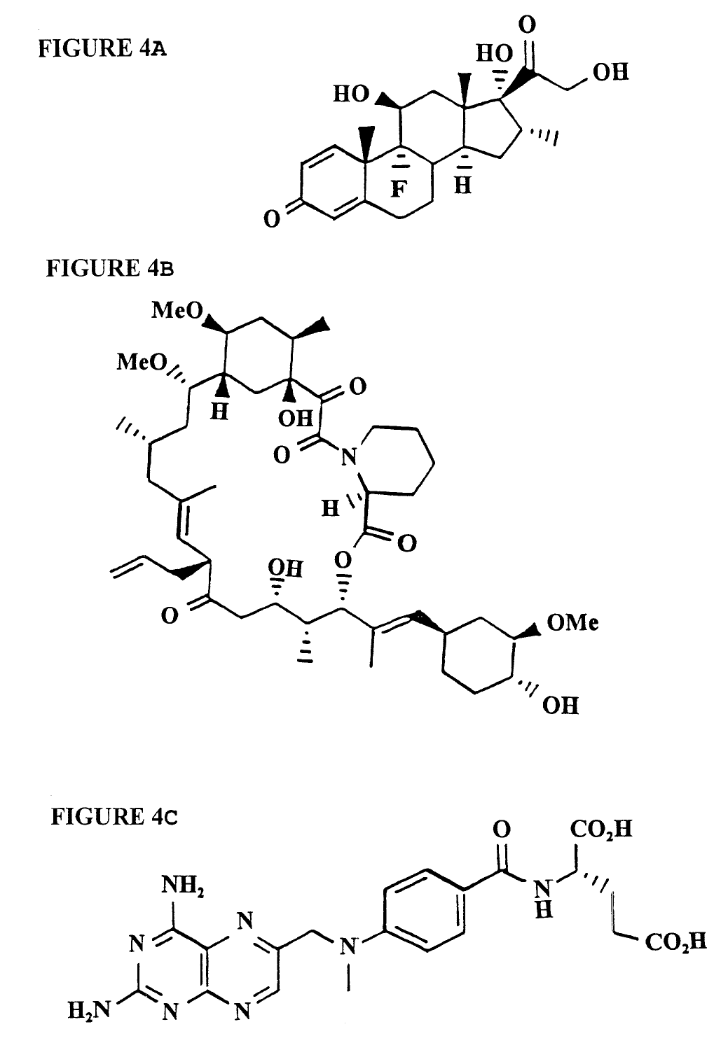

Dexamethasone (DEX) is an attractive ligand half (also referred to as “chemical handle”) (FIG. 4A). DEX binds rat glucocorticoid receptor (rGR) with a K_{D} of 5 nM, (14) can regulate the in vivo activity and nuclear localization of rGR fusion proteins (15), and is commercially available. Affinity columns for rGR have been prepared via the C₂₀ ∝-hydroxy ketone of dexamethasone (16, 17).

The antibacterial and anticancer drug methotrexate (MTX) is used in place of FK506 (FIG. 4C, 4B). FK506 is not available in large quantities, coupling via the C₂₁ allyl group requires several chemical transformations including silyl protection of FK506, (18, 19) and FK506 is both acid and base-sensitive. MTX, on the other hand, is commercially available and can be modified selectively at its γ-carboxylate without disrupting dihydrofolate reductase (DHFR) binding (20, 21). Even though MTX inhibits DHFR with pM affinity, (21) both E. coli and S. cerevisiae grow in the presence of MTX when supplemented with appropriate nutrients (22).

For example, the ability of DEX-MTX to mediate the dimerization of LexA-rGR and B42-DHFR was shown by WO 01/53355 based on lacZ transcription and that both DEX and MTX uncoupled, can, competitively disrupt this dimerization.

Other ligand halves may be for example, steroids, such as the Dexamethasone used herein; enzyme inhibitors, such as Methotrexate used herein; drugs, such as KF506; hormones, such as the thyroid hormone 3,5,3′-triiodothyronine (structure below)

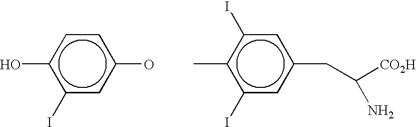

Ligands for nuclear receptors, such as retinoic acids, for example the structure below

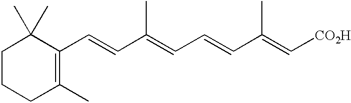

General cofactors, such as Biotin (structure below)

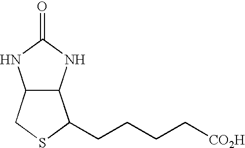

and antibiotics, such as Coumermycin (which can be used to induce protein dimerization according to Perlmutter et al., Nature 383, 178 (1996)).

A commercial source of traditional, non-covalent dimeric molecules for use in a chemically induced dimerization system is ARIAD (www.ariad.com), who call their CID “ARGENT TECHNOLOGY.” The mentioned compounds as well as the commercial compounds can be derivatized for use in the eACID system. Specifically, one of the ligand halves is a substrate of the “assisting” enzyme, which binds with its corresponding receptor domain in the presence of the “assisting” enzyme.

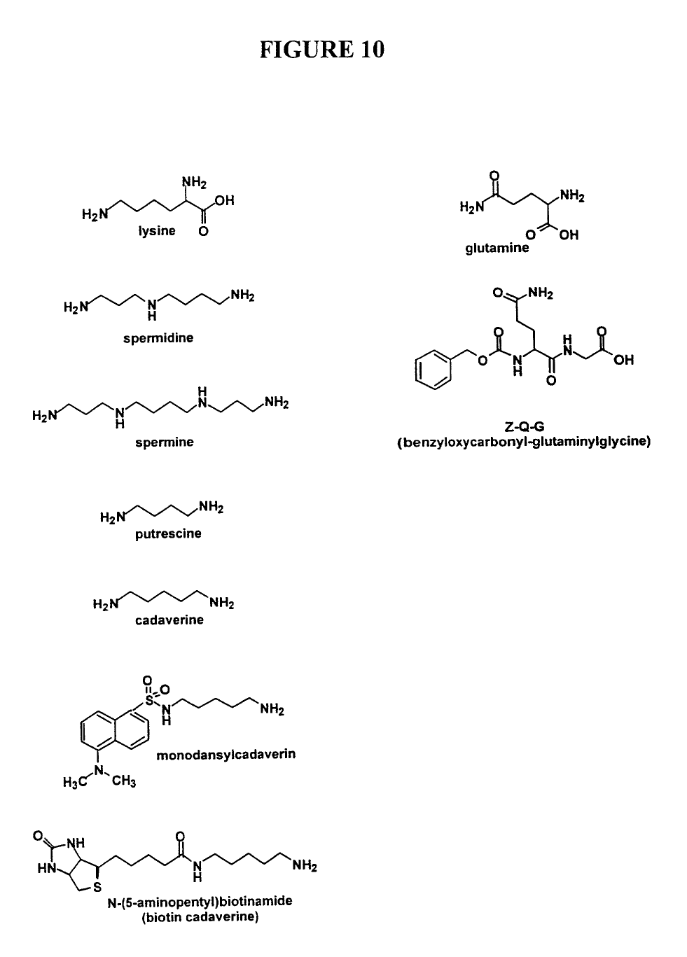

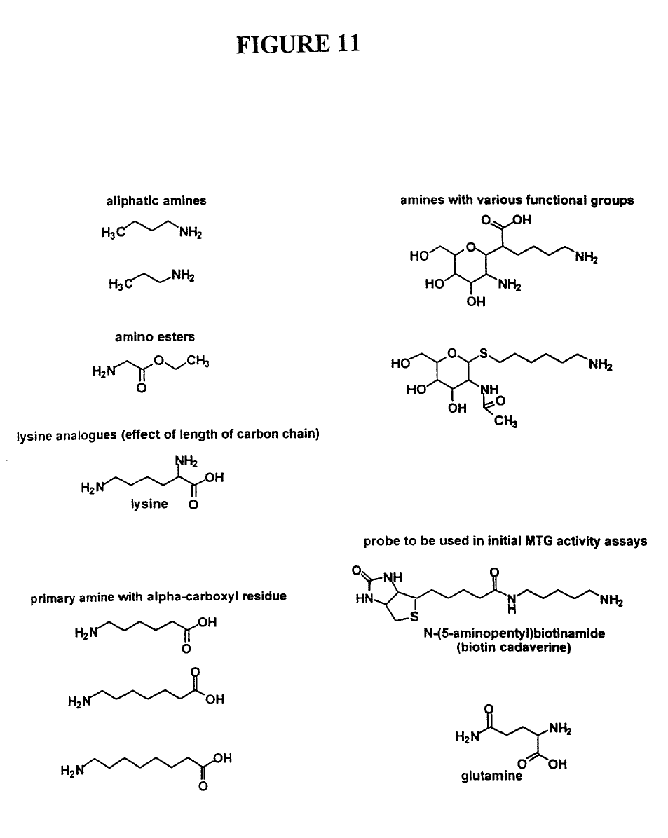

Examples of substrates which can be used with a transglutaminase enzyme are shown in FIGS. 10 and 11. Once dimerized with another ligand half, each one of the shown substrates can be used in the eACID system to screen proteins having transglutaminase activity.

Linkage of the Ligand Halves in the Dimeric Small Molecule

While the ligand halves can be simply linked by a covalent bond between the two of them, more elaborate linkages may also be used depending on the screen to be performed. The linkage may be formed by any of the methods known in the art. For example, Jerry March, Advanced Organic Chemistry (1985) Pub. John Wiley & Sons Inc; and HH, House, Modern Synthetic Reactions (1972) pub. Benjamin Cummings. Descriptions of linkage chemistries are also provided by WO 94/18317, WO 95/02684, WO 96/13613, WO96/06097, and WO 01/53355, these references being incorporated herein by reference.

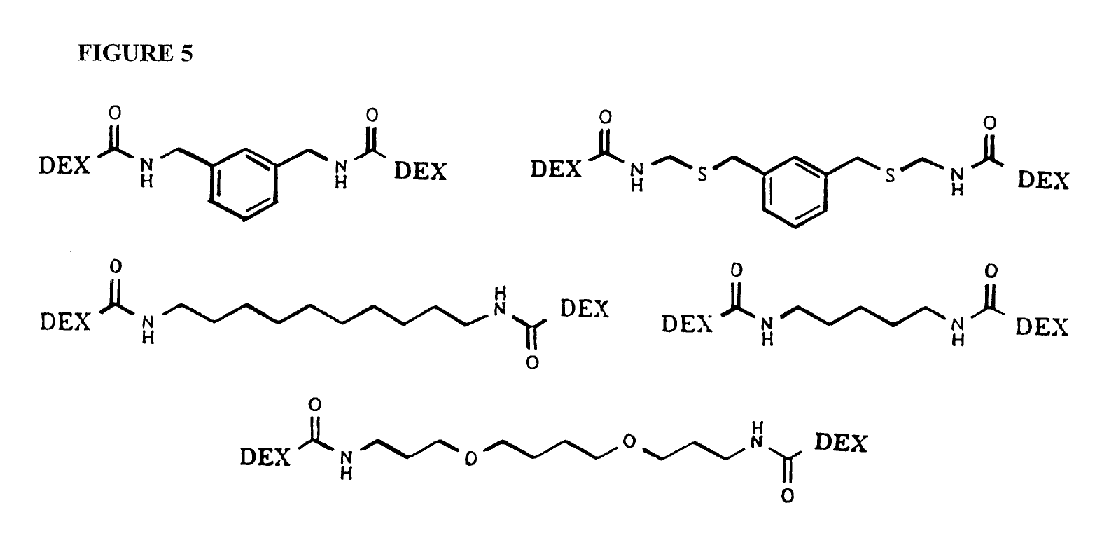

As an illustrative example of alternative ways of linking the ligand halves, several of the DEX-DEX compounds that have been synthesized to date are shown in FIG. 5. The linkers are all commercially available or can be prepared in a single step. The linkers vary in hydrophobicity, length, and flexibility.

“Assisting” Enzyme

The element of an “assisting” enzyme is specific to the eACID system. The enzymes may be known enzymes or novel proteins which are being screened for specific enzymatic activity. Novel enzymes can be evolved using combinatorial techniques.

Once a desired substrate is selected and formed into the dimeric small molecule, a large number of enzymes and derivatives of enzymes can be screened. A variety of enzymes and enzymes classes are listed on the World Wide Web beginning at prowl.rockefeller.edu/enzymes/enzymes.htm. All enzymes are given an Enzyme Commission (E.C.) number allowing it to be uniquely identified. E.C. numbers have four fields separated by periods, “a.b.c.d”. The left-hand-most field represents the most broad classification for the enzyme. The next field represents a finer division of that broad category. The third field adds more detailed information and the fourth field defines the specific enzyme. Thus, in the “a” field the classifications are oxidoreductases, transferases, hydrolases, lyases, isomerases, and ligases. Each of these “a” classifications are then further separated into corresponding “b”, each of which in turn is separated into corresponding “c” classifications, which are then further separated into corresponding “d” classes.

The classes that have particular applicability to the described eACID system are transferases, lyases and ligases.

The subclasses of transferases are, for example:

2.1 one carbon, 2.2 aldehydes or ketones, 2.3 acyl, 2.4 glycosyl, 2.5 alkyl or aryl, 2.6 N-containing, 2.7 P-containing, 2.8 S-containing, and 2.9 Se-containing.

The subclasses of lyases are, for example:

4.1 C-C, 4.2 C-O, 4.3 C-N, 4.4 C-S, 4.5 C-halide, and 4.6 P-O.

The subclasses of ligases are, for example:

6.1 C-O, 6.2 C-S, 6.3 C-N, 6.4 C-C, and 6.5 P-ester.

Each of the mentioned classes is further separated into sub,

sub-classes, i.e. the “c” level, and then the “d” level.

Transglutaminases and kinases are particularly useful in the described methods.

Moreover, new enzymes are discovered and are intended to be included within the scope of this invention, which is itself designed to evolve or discover such new enzymes.

Design of the Protein Chimeras

The second important feature is the design of the protein chimeras. The protein chimeras based on the yeast two-hybrid assay were chosen because of its flexibility. Specifically, the Brent two-hybrid system is used, which uses LexA as the DNA-binding domain and B42 as the transcription activation domain. The Brent system is one of the two most commonly used yeast two-hybrid systems. An advantage of the Brent system is that it does not rely on Gal4 allowing use of the regulatable Gal promoter. lacZ under control of 4 tandem LexA operators are used as the reporter gene. For example, simple LexA-rGR and DHFR and B42-rGR and DHFR fusion proteins that do not depart from the design of the Brent system have been made. In the Brent system, the full length LexA protein which includes both the N-terminal DNA-binding domain and the C-terminal dimerization domain is used. The B42 domain is a monomer. The C-terminal hormone-binding domain of the rat glucocorticoid receptor was chosen because this domain was shown to work previously in the yeast three-hybrid system reported by Licitra, et al. Both the E. coli and the murine DHFRs are used because these are two of the most well characterized DHFRs. The E. coli protein has the advantage that methotrexate binding is independent of NADPH binding.

The protein chimeras can be varied in four ways: (1) invert the orientation of the B42 activation domain and the receptor; (2) introduce tandem repeats of the receptor; (3) introduce (GlyGlySer)ₙ linkers between the protein domains; (4) vary the DNA-binding domain and the transcription activation domain. Additional detail about previous systems can be found in WO 01/53355.

Design of Reporter Genes

A reporter gene assay measures the activity of a gene's promoter. It takes advantage of molecular biology techniques, which allow one to put heterologous genes under the control of a mammalion cell (23, 24). Activation of the promoter induces the reporter gene as well as or instead of the endogenous gene. By design the reporter gene codes for a protein that can easily be detected and measured. Commonly it is an enzyme that converts a commercially available substrate into a product. This conversion is conveniently followed by either chromatography or direct optical measurement and allows for the quantification of the amount of enzyme produced.

Reporter genes are commercially available on a variety of plasmids for the study of gene regulation in a large variety of organisms (24). Promoters of interest can be inserted into multiple cloning sites provided for this purpose in front of the reporter gene on the plasmid (25, 26). Standard techniques are used to introduce these genes into a cell type or whole organism (e.g., as described in Sambrook, J., Fritsch, E. F. and Maniatis, T. Expression of cloned genes in cultured mammalian cells. In: Molecular Cloning, edited by Nolan, C. New York: Cold Spring Harbor Laboratory Press, 1989). Resistance markers provided on the plasmid can then be used to select for successfully transfected cells.

Ease of use and the large signal amplification make this technique increasingly popular in the study of gene regulation. Every step in the cascade DNA→RNA→Enzyme→Product→Signal amplifies the next one in the sequence. The further down in the cascade one measures, the more signal one obtains.

In an ideal reporter gene assay, the reporter gene under the control of the promoter of interest is transfected into cells, either transiently or stably. Receptor activation leads to a change in enzyme levels via transcriptional and translational events. The amount of enzyme present can be measured via its enzymatic action on a substrate.

In addition to the reporter genes mentioned above, ura3, which encodes orotidine-5′-phosphate decarboxylase and is required for uracil biosynthesis, can be used as the reporter gene. Ura3 has the advantage that it can be used both for positive and negative selections-positive for growth in the absence of uracil and negative for conversion of 5-fluoroorotic acid (5-FOA) to 5-fluorouracil, a toxic byproduct. Cleavage of the glycosidic bond and disruption of ura3 transcription is selected for based on growth in the presence of 5-FOA. The advantage to the 5-FOA selection is that the timing of addition of both the dimeric small molecule and 5-FOA can be controlled.

Host Cell

The host cell for the foregoing screen may be any cell capable of expressing the protein or cDNA library of proteins to be screened. Some suitable host cells have been found to be yeast cells, such as Saccharomyces Cerevisiae, and bacterial cells, such as E. Coli.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

### Example 1

Transglutaminase (“TG”) Assisted CID.

The protein modification system calls for three modifications to the basic CID system(1):

i) transglutaminase (TG), an enzyme that catalyzes the formation of a peptide linkage between a peptide bound glutamine residue and an amine, is included in the system;

ii) one of the receptor domains is replaced with a protein that contains a specific TG recognition sequence; and

iii) one of the linked ligands is replaced with an amine that can act as a TG substrate.

The TG catalyzes the formation of a peptide linkage between the TG recognition sequence and the amine of the small-molecule ligand; the resultant complex leads to protein dimerization and hence a cellular read-out.

Components of TG-ACID system:

1) Reporter Plasmid: The reporter plasmid that is being used in the initial eACID system (pMW106) is identical to that used in the WO 01/53355 and consists of 8 LexA operators (DNA binding sites recognized by the DNA-binding domain of the LexA protein) and a lacZ reporter gene (1). Binding of the reconstituted reporter protein at the LexA DNA binding site results in transcription of the lacZ gene. This yields an easily detectable cellular readout. Reporter plasmids that contain different numbers of LexA operators (and that therefore differ in their degree of sensitivity) are also employed.

2) Receptor/Transcription Factor 1 (fusion protein 1): RTF protein 1 is identical to that used in the Cornish CID system and consists of the B42 transcriptional activation domain fused to bacterial dihydrofolate reductase (DHFR) (1).

3) Receptor/Transcription Factor 2 (fusion protein 2): RTF protein 2 consists of LexA fused to a “scaffold” protein, in this case a catalytically inactive version of staphylococcal nuclease (SNase) that has been constructed to contain a microbial TG substrate sequence. The SNase is being used as a TG substrate presentation platform because it folds spontaneously without chaperones, has a prominently exposed loop on its surface that can be used to present a peptide sequence to other cellular proteins, and can be strongly expressed in eukaryotes (3). NOTE: The designations Receptor/Transcription Factor Protein 1 and Receptor/Transcription Factor Protein 2 are somewhat arbitrary. That is, as the chimeric proteins are modular by design (contain both receptor/substrate and transcription factor components), they may be “mixed and matched” with one another and tested in all possible combinations. Thus, although a specific chimera has been labeled as “1” or “2”, this is only for the sake of simplicity.

4) Small molecule substrate: The small molecule substrate consists of two halves: 1-a ligand of DHFR (methotrexate (MTX)), and 2-a ligand (or substrate) of TG (an amine).

5) Transglutaminase (“TG”) enzyme: The TG gene has been cloned from the Streptoverticillium mobaraense and Streptoverticillium cinnamoneum bacteria and is under the control of an inducible promoter. Tissue TG and FXIIIa TG have also been cloned for use in the eACID system.

Small Molecule Substrate (MP5)—Synthesis and Cell Permeability

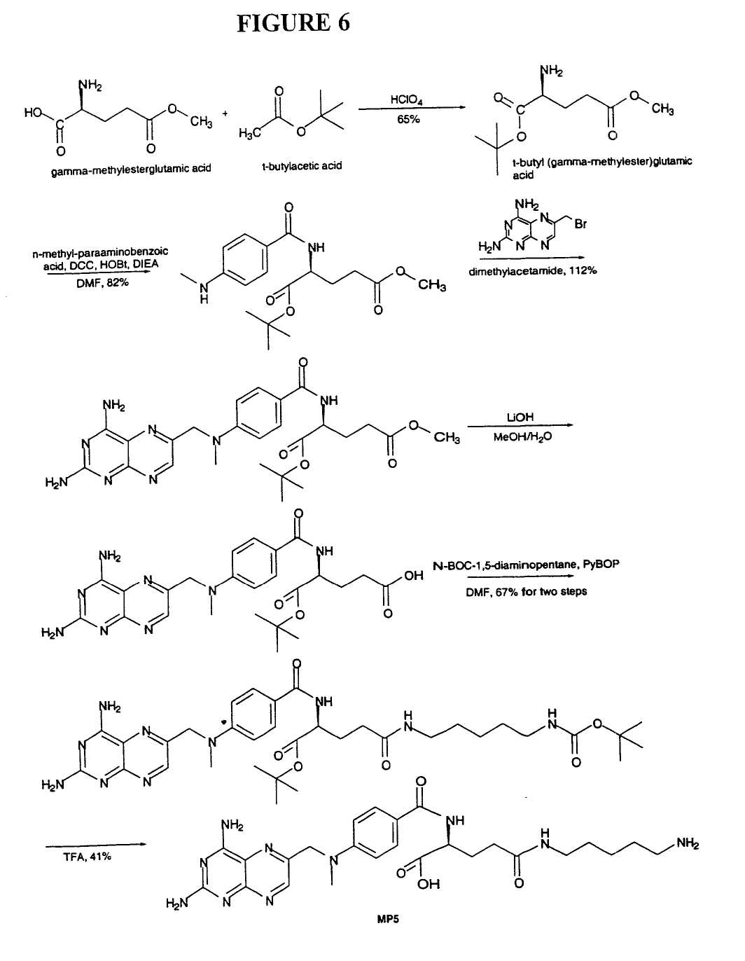

The small molecule substrate consists of two recognition domains; one domain binds dihydrofolate reductase (DHFR) and the other is utilized as a nucleophile by TG. The small molecule is cell-permeable, and is not excreted from the cell. The first small molecule consists of MTX (a synthetic folate analogue that binds DHFR with nM affinity) linked to an aminopentane (a substrate of MTG (4)). Synthesis of the small molecule required six steps from commercial/lab materials (see FIG. 6). All intermediates and the final product were purified by silica-gel chromatography and characterized by nuclear magnetic resonance (NMR) spectroscopy and fast-atom bombardment mass spectrometry (MS).

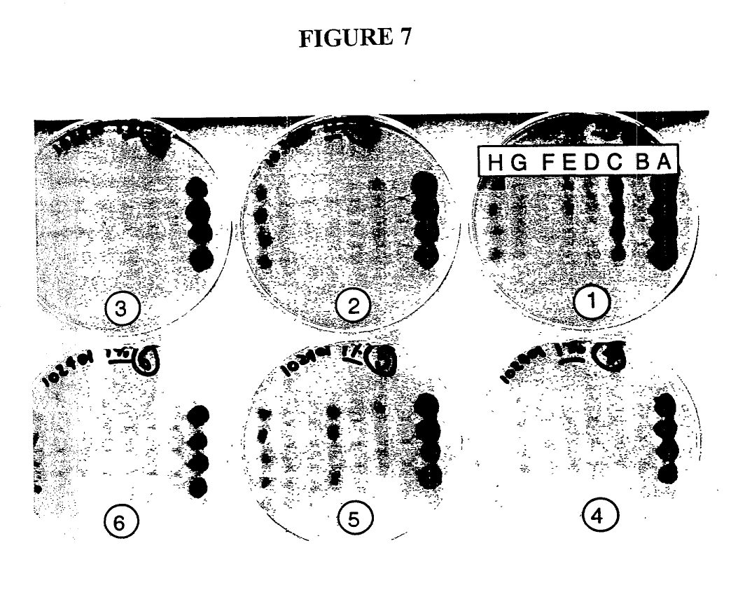

To demonstrate that the MP5 dimerizer was both able to enter the yeast cell and also act as a substrate for DHFR a small molecule competition assay was performed. That is, performing a Y3H assay (using a small molecule that has already been demonstrated to be both cell permeable and a DHFR substrate) using MP5 as a competitor molecule. This competition assay was performed using D8M as the “well characterized” small molecule. The results shown in FIG. 7 clearly demonstrate that MP5 is cell permeable and that it can compete with D8M for binding to the DHFR fusion protein in vivo.

D8M has the structure:

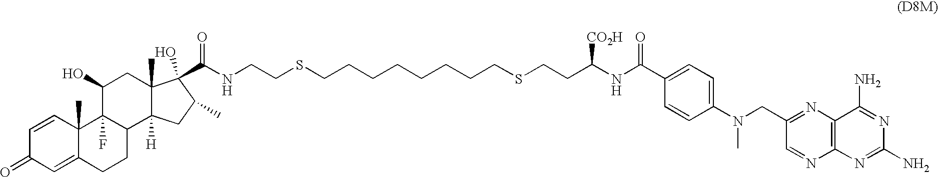

Scaffold Protein Containing TG Substrate Recognition Sequence—Construction of Receptor Fusions and Expression in Yeast

The basic CID system (1) consists of a fusion protein that contains a DNA-binding protein (LexA) and the rat glucocorticoid receptor (rGR). Conversion of this basic CID system into an eACID system requires the substitution of the rGR with a presentation protein (such as SNase) that contains a TG substrate recognition sequence. A number of SNase constructs have been engineered that contain the MTG substrate recognition sequence in the exposed loop. Based on these, genes that code for receptor fusion constructs have also been constructed.

Based on the published data, and especially reports from Ajinomoto (4), four substrate recognition sequences were constructed into a biologically inert version of SNase. The four sequences are:

i) LGQG

ii) LQGG

iii) LLQG

iv) LGGG

The first three sequences are substrates for TG modification; the forth sequence is a control sequence that is not recognized nor modified by the TG. All four constructs have been made and transformed into E. coli; frozen stocks and miniprep DNA have been made and are in lab.

Using the above SNase constructs and other lab constructs, plasmids coding for LexA-SNase fusions have been engineered and transformed into E. coli; frozen stocks and miniprep DNA have been made and are in lab (strains [V770E, V776E]).

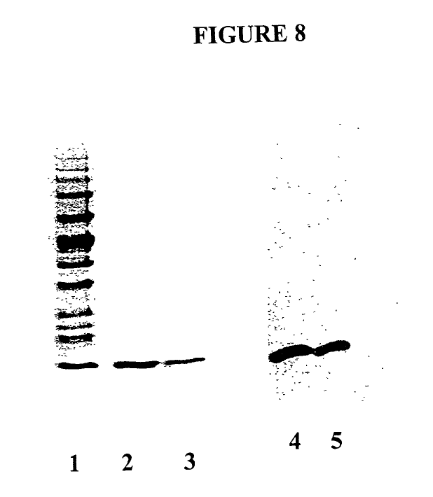

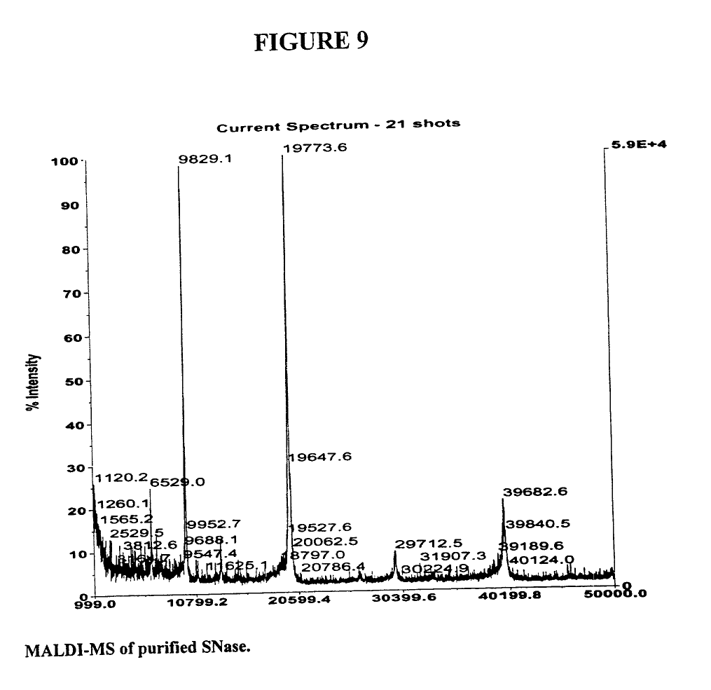

Snase clones were transformed into Escherichia coli and then into Saccharomyces cerevisiae (yeast) (FY250). Yeast containing the SNase clone were grown and harvested, and SNase was purified using a Ni-affinity column. Purified SNase (single band on a Coomassie stained gel, see FIG. 8) was analyzed using MS. The expected molecular weight for SNase is ˜20,017 Da; a peak at 19,774 Da is likely from SNase. See FIG. 9. The difference in expected molecular weight (244 Da) corresponds to the molecular weight of two amino acids (assuming amino acid average molecular weight to be 114 Da). This peak is very strong (relative to background) and is well resolved from other signals.

These results demonstrate the use of MS to identify purified SNase. Further, this allows one to theorize that this approach may be successful in the detection and identification of TG-mediated post translational modification of a target protein (in this example SNase).

Subcloning of Microbial Transglutaminase (S.mobaraense)—Expression in Yeast and Activity Assays

In an effort to address the reasonable possibility that the TG substrate sequence on the SNase protein may function, function better, or function only when fused to the B42 activation domain (instead of the LexA DNA binding domain), B42 fusions were made as well. Plasmids coding for B42-SNase fusions have been constructed and transformed into E. coli; frozen stocks and miniprep DNA have been made and are in lab (strains [V762E, V769E].

| Plasmid on | | TG substrate | Strain name | Strain name |
|---|---|---|---|---|
| which construct is based | Fusion protein | sequence | (Bacterial/ TG₁) | (Yeast/FY₂₅₁) |
| pEG₂₀₂ | LexA-SNase | LLQG | V₇₇₀E | See 80601* |
| pEG₂₀₂ | LexA-SNase | LLQG | NYM* | NYM** |
| pEG₂₀₂ | LexA-SNase | LQGG | NYM* | NYM** |
| pEG₂₀₂ | LexA-SNase | LGQG | NYM* | NYM** |
| pJG₄₋₅ | B42-SNase | LLQG | V₇₆₂E | NYM** |
| pJG₄₋₅ | B42-SNase | LQGG | V₇₉₄E | NYM** |
| pJG₄₋₅ | B42-SNase | LGQG | NYM* | NYM** |
| pJG₄₋₅ | B42-SNase | LGGG | NYM* | NYM** |

*Patches made but have not named strain nor made frozen stocks.
**NYM (not yet made)

Three of the eight proposed constructs have been made (see Table) and tested. Based on the success wiht the three constructs made, the other constructs are as expected to work. Early constructs and experiments with those constructs were based on TG from both S. mobaraense and S. cinnamoneum. However, other are available.

Transglutaminase (TG) was chosen as the enzyme that would be used to catalyze the covalent linking of a small molecule to the target protein. This group of enzymes catalyzes the post-translational modification of proteins leading to the formation of a peptide linkage between the g-carboxamide group of a peptide-bound glutamine residue and the primary amino group of either a peptide-bound lysine or polyamine. The resultant peptide bonds are covalent, stable, and resistant to proteolysis (27). We considered 10 of the most well characterized TGs. Their properties are compared and contrasted in Table 1.

TABLE 1
Comparison of Transglutaminases

| TG | Oligomerization | Ca⁺⁺ | | |
|---|---|---|---|---|
| Name | State | Requirement | Clone | Comments^{a, b, c, d} |
| Factor XIIIa | Heterotetramer | Yes | (11, 12)^{c} | Zymogen (activated by the protease Thrombin) |
| Tissue | Monomer | Yes | (23-26) | No protease activation althrough active, may |
| | | | | be present intracellularly in and inactive form |
| Keratinocyte | Monomer | Yes | (37-38)^{c} | No protease activation |
| Epidermal | Monomer | Yes | (42)^{c} | Protease activation; not well characterized |
| Hair follicle | Homodimer | Yes | None | Probably a variant of Epidermal TG, but |
| | | | | possibly a distinct gene product; |
| | | | | immunochemically distinct from Epidermal TG |
| Prostate | Homodimer | Yes | (44)^{c} | Very poorly understood |
| Band 4.2 | Monomer | No | (48)^{c} | Within erythrocyte plasma membrane; |
| | | | | catalytically inactive (since has A in place of |
| | | | | C in active site) |
| Hemocyte | Monomer | Unclear | (52, 53, 54)^{c} | Anthropod analogues of Factor XIIIa and |
| and Annulin | | | | Keratinocyte TG; may be post translationally |
| | | | | modified; Hemocyte TG does not require |
| | | | | proteolytic cleavage for activation |
| Plant | Unclear | No | None | Very ill defined |
| Microbial | Monome | No | YES^{c} | Unclear if covered Ajimomoto Patent(s) |

^{a}FASEB, Rice et al., 5: 3071; 1991
^{b}JBC, Davie et al., 265: 13411; 1990
^{c}Thrombosis and Haemotasis, Paulsson et al., 71(4): 402; 1994
^{d}JBC, Shimonishi et al., 268: 11565; 1993 (Streptoverticillium sp.)
^{e}Biochimie, Duran et al., 80: p. 313; 1998

Detect and Quantify Transglutaminase Activity

The calorimetric assay is reasonably well established and has been performed in one form or another in a number of different labs using a number of different sources and preparations of TG (5, 6). In the calorimetric assay, the substrate 5-(biotinamido)pentylamine (BAP) is covalently incorporated into N,N′-dimethylcasein (DMC) via a TG-dependent process. This biotinylated product is detected by the addition of streptavidin-alkaline phosphatase (AP) and quantitated by adding p-nitrophenyl phosphate and measuring absorbance at 405 nm TG (5, 6). This type of assay has successfully been used to detect the activity of a variety of TG samples including recombinant factor XIIIa in crude E. coli lysate (6).

The colorimetric assay was performed a number of times, testing both a positive control (purchased purified tissue TG) and various crude soluble yeast extracts that contained plasmids coding for various versions of microbial TG, detecting TG activity.

Subcloning of Factor XIIIA Human Transglutaminase

In important aspect of the eACID screen is the ability to express an enzyme that is able to form a covalent linkage between the small molecule ligand and a target sequence. TG has the ability to perform this task. Microbial TG has been cloned and used in a number of preliminary experiments. Toxicity assays indicate that MTG is active. Thus, an alternate TG enzymes be tested. Two alternate TG enzymes were selected-tissue TG and factor XIIIa.

Factor XIII is responsible for cross-linking fibrin chains during blood clotting and is involved in wound healing and tissue repair. Plasma FXIII is composed of two subunits, A and B; A is responsible for catalytic activity whereas B acts as a carrier protein that “protects” the A subunits.

Intracellular FXIII in platelets and monocytes is composed of only A subunits (7). Board et al. have demonstrated that expression of recombinant FXIII subunit A in yeast can yield enzymatic activity in fresh yeast lysates (7-10). This is desirable in this screen. Plasmids expressing FXIIIa were obtained from Board (pRB334 and pYF13AH (7-10) and strains containing these plasmids were constructed. These are tested for TG activity. Board et al. also published an interesting report that involved the use of a ubiquitin-FXIIIa fusion that also yielded active FXIIIa in crude yeast extracts (7).

X-gal Screens Using All Components of eACID System

Initial screens using all the components of the eACID system yield results showing small molecule dependent activation of the reported gene.

Discussion

The SNase scaffold protein has been successfully used to present a peptide sequence within a cell (3). A promising alternate scaffold is the thioredoxin protein which has been used as a peptide presentation protein in yeast 2 hybrid assays (11). Another approach to peptide presentation would be to simply fuse the TG substrate sequence directly to the LexA (or B42) domain. A similar approach was taken by Fields in a yeast 2 hybrid assay (12). Further, the crystal structure of LexA has recently been published (13), and this will likely make the rational design of any LexA fusion constructs much easier.

The choise of a presented protein, SNase in this case, should take into account the cell type specific endogenous factors that can contribute to activation of the reporter. If background “noise” is found to be too high to tolerate, a less sensitive reporter construct can be used. Alternatively, the MALDI-MS can be used to identify other targets of a TG and account for their interference in the system. This can be done by co-expressing both enzyme and target in cells that are growing in the presence of a TG substrate small molecule (such as MP5 etc.), followed by purification of the target and subjecting it to MS analysis. A more straight forward assay would be to express and purify both TG and target protein, allow cross-linking to occur in vitro, then performing MS analysis.

Bibliography

1a. U.S. Pat. No. 5,468,614, and Yang et al., Nucleic Acid Research 1995, 23, 1152-1156

1b. U.S. Pat. No. 5,928,868, and Licitra, Edward J., et al., PNAS, USA 93, 1996, 93, 12817-12821.

1. H. Lin, W. Abida, R. Sauer, V. Cornish, J. Am. Chem. Soc. 2000, 122, 4247-4248.

2. S. J. Kopytek, R. F. Standaert, J. C. Dyer, J. C. Hu, Chem Biol 2000, 7, 313-21.

3. T. C. Norman, D. L. Smith, P. K. Sorger, B. L. Drees, S. M. O'Rourke, T. R. Hughes, C. J. Roberts, S. H. Friend, S. Fields, A. W. Murray, Science 1999, 285, 591-5.

4. T. Ohtsuka, A. Sawa, R. Kawabata, N. Nio, M. Motoki, J Agric Food Chem 2000, 48, 6230-3.

5. W. M. Jeon, K. N. Lee, P. J. Birckbichler, E. Conway, M. K. Patterson, Jr., Anal Biochem1989, 182, 170-5.

6. T. F. Slaughter, K. E. Achyuthan, T. S. Lai, C. S. Greenberg, Anal Biochem 1992, 205, 166-71.

7. M. Coggan, R. Baker, K. Miloszewski, G. Woodfield, P. Board, Blood 1995, 85, 2455-60.

8. P. G. Board, K. Pierce, M. Coggan, Thromb Haemost 1990, 63, 235-40.

9. Kangsadalampai, P. G. Board, Blood 1998, 92, 2766-70.

10. S. Kangsadalampai, G. Chelvanayagam, R. T. Baker, P. Yenchitsomanus, P. Pung-amritt, C. Mahasandana, P. G. Board, Blood 1998, 92, 481-7.

11. P. Colas, B. Cohen, T. Jessen, I. Grishina, J. McCoy, R. Brent, Nature 1996, 380, 548-50.

12. M. Yang, Z. Wu, S. Fields, Nucleic Acids Res 1995, 23, 1152-6.

13. Y. Luo, R. A. Pfuetzner, S. Mosimann, M. Paetzel, E. A. Frey, M. Cherney, B. Kim, J. W. Little, N. C. Strynadka, Cell 2001, 106, 585-94.

14. Chakraborti, P.; Garabedian, M.; Yamamoto, K.; S S Simons, J. J. Biol. Chem. 1991, 266, 22075-22078.

15. Picard, D.; Yamamoto, K. EMBO J. 1987, 6, 3333-3338.

16. Govindan, M.; Manz, B. Eur. J. Biochem. 1980, 108, 47-53.

17. Manz, B.; Heubner, A.; Kohler, I.; Grill, H.-J-.; Pollow, K. Eur. J. Biochem. 1983, 131, 333-338.

18. Spencer D M, et al., Curr Biol. Jul. 1, 1996 6(7): 839-47.

19. Pruschy, M.; Spencer, D.; Kapoor, T.; Miyake, H.; Crabtree, G.; Schreiber, S. Chem. Biol. 1994, 1, 163-172.

20. Kralovec, J.; Spencer, G.; Blair, A.; Mammen, M.; Singh, M.; Ghose, T. J. Med. Chem. 1989, 32, 2426-2431.

21. Bolin, J.; Filman, D.; Matthews, D.; Hamlin, R.; Kraut, J. J. Biol. Chem. 1982, 257, 13663-13672.

22. Huang, T.; Barclay, B.; Kalman, T.; vonBorstel, R.; Hastings, P. Gene 1992, 121,167-171.

23. Gorman, C. M. et al., Mol. Cell Biol. 2: 1044-1051 (1982).

24. Alam, J. and Cook, J. L., Anal. Biochem. 188: 245-254, (1990).

25. Rosenthal, N., Methods Enzymo. 152: 704-720 (1987).

26. Shiau, A. and Smith, J. M., Gene 67: 295-299 (1988).

27. Greenberg, C. S., Birckbichler, P. J., and Rice, R. H. Faseb J 5, 3071-7 (1991).

28. Park S H, Raines R T, Nat Biotechnol. 2000 August; 18(8):847-51.

What is claimed is:

1. A method for identifying which protein from a pool of candidate proteins catalyzes in a cell a bond forming reaction between a first substrate and a second substrate, comprising:
(a) providing a dimeric small molecule which comprises a known moiety that binds a known receptor domain covalently linked with a moiety that contains the first substrate;
(b) introducing the dimeric molecule into a cell which comprises
i) a first fusion protein comprising the known receptor domain,
ii) a second fusion protein comprising the second substrate,
iii) a protein from the pool of candidate proteins, and
iv) a reporter gene wherein expression of the reporter gene is conditioned on the proximity of the first fusion protein to the second fusion protein;
(c) permitting the dimeric molecule to bind to the first fusion protein and to enzymatically form a bond with the second fusion protein so as to activate the expression of the reporter gene;
(d) selecting which cell expresses the reporter gene; and
(e) identifying the protein that catalyzes the bond formation reaction in the cell between the first substrate and the second substrate.

2. The method of claim 1, wherein the protein is encoded by a DNA from the group consisting of genomic DNA, cDNA and synthetic DNA.

3. The method of claim 1, wherein the pool of candidate proteins is obtained by combinatorial techniques.

4. The method of claim 1, wherein the steps (b)-(e) of the method are iteratively repeated in the presence of a preparation of random proteins for competitive enzymatic bond formation so as to identify a protein having enhanced enzymatic activity.

5. The method of claim 1, wherein the cell is an insect cell, a yeast cell, a bacterial cell, or a mammalian cell.

6. The method of claim 1, wherein the cell is a yeast cell.

7. The method of claim 1, wherein the first fusion protein further comprises a DNA binding domain, and the second fusion protein further comprises a transcription activation domain.

8. The method of claim 1, wherein the first fusion protein further comprises a transcription activation domain, and the second fusion protein further comprises a DNA binding domain.

9. The method of claim 7 or 8, wherein the DNA-binding domain is LexA, Gal4 or VP16.

10. The method of claim 7 or 8, wherein the transcription activation domain is B42.

11. The method of claim 1, wherein the known moiety that binds a known receptor domain is a Methotrexate moiety, a dexamethasone moiety, FK506 moiety, an FK506 analog, a teracycline moiety, or a cephem moiety.

12. The method of claim 1, wherein the known receptor domain is that of dihydrofolate reductase (“DHFR”), glucocorticoid receptor, FKBP12, FKBP mutants, tetracycline repressor, or a penicillin binding protein.

13. The method of claim 12, wherein the DHFR is the E.coli DHFR (“eDHFR”).

14. The method of claim 1, wherein the first fusion protein is eDHFR-LexA or R61-LexA.

15. The method of claim 1, wherein the first fusion protein is eDHFR-B42 or R61-B42.

16. The method of claim 1, wherein the reporter gene is Lac Z, ura 3, GFP, β-lactamase, luciferase or an antibody coding region.

17. The method of claim 1, wherein the reporter gene is Lac Z.

18. The method of claim 1, wherein the first substrate is an amine.

19. The method of claim 1, wherein the second substrate is an amine.

20. The method of claim 1, wherein the second substrate is an amino acid sequence containing a lysine.

21. The method of claim 1, wherein the second substrate is an amino acid sequence containing a glutamine.

22. The method of claim 1, wherein the second substrate is an amino acid sequence containing-leucine-glycine-glutamine-glycine-.

23. The method of claim 1, wherein the second substrate is an amino acid sequence containing-leucine-glutamine-glycine-glycine-.

24. The method of claim 1, wherein the second substrate is an amino acid sequence containing-leucine-leucine-glutamine-glycine-.

25. The method of claim 1, wherein the second substrate is a modified staphylococcal nuclease (“SNase”) or a modified thioredoxin containing an amino acid sequence containing a glutamine.

26. The method of claim 1, wherein the protein that catalyzes bond formation is a transglutaminase.

27. The method of claim 1, wherein the protein that catalyzes bond formation is a microbial transglutaminase, a tissue transglutaminase, or Factor XIIIA.

28. The method of claim 1, wherein the dimeric small molecule has the structure: 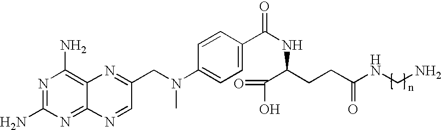
wherein n is an integer from 1 to 20.

29. The method of claim 28, wherein n is an integer from 2 to 12.

30. The method of claim 28, wherein n is an integer from 3 to 9.

31. The method of claim 28, wherein n is 5.

32. A new protein cloned by the method of claim 1.

33. A method for identifying which substrate from a pool of candidate substrates is selected in a cell by a known enzyme for a bond forming reaction between the substrate and a known amino acid, comprising:
(a) providing a dimeric small molecule which comprises the substrate covalently linked to a moiety known to bind a receptor domain;
(b) introducing the dimeric molecule into a cell which comprises
i) a first fusion protein comprising the receptor domain,
ii) a second fusion protein comprising the known amino acid,
iii) the known enzyme, and
iv) a reporter gene wherein expression of the reporter gene is conditioned on the proximity of the first fusion protein to the second fusion protein;
(c) permitting the dimeric molecule to bind to the first fusion protein and to enzymatically form a bond with the second fusion protein so as to activate the expression of the reporter gene;
(d) selecting which cell expresses the reporter gene; and
(e) identifying the substrate selected by the known enzyme in the cell for the bond forming reaction between the substrate and the known amino acid.

34. The method of claim 33, the pool of candidate substrates is obtained by combinatorial techniques.

35. The method of claim 33, wherein the steps (b)-(e) of the method are iteratively repeated in the presence of a preparation of random substrates for competitive enzymatic bond formation so as to identify a substrate competitively selected by the known enzyme.

36. The method of claim 33, wherein the cell is an insect cell, a yeast cell, a bacterial cell, or a mammalian cell.

37. The method of claim 33, wherein the cell is a yeast cell.

38. The method of claim 33, wherein the first fusion protein further comprises a DNA binding domain, and the second fusion protein further comprises a transcription activation domain.

39. The method of claim 33, wherein the first fusion protein further comprises a transcription activation domain, and the second fusion protein further comprises a DNA binding domain.

40. The method of claim 38 or 39, wherein the DNA-binding domain is LexA, Gal4 or VP16.

41. The method of claim 38 or 39, wherein the transcription activation domain is B42.

42. The method of claim 33, wherein the moiety known to bind a receptor domain is a Methotrexate moiety, a dexamethasone moiety, FK506 moiety, an FK506 analog, a teracycline moiety, or a cephem moiety.

43. The method of claim 33, wherein the receptor domain is that of dihydrofolate reductase (“DHFR”), glucocorticoid receptor, FKBP12, FKBP mutants, tetracycline repressor, or a penicillin binding protein.

44. The method of claim 43, wherein the DHFR is the E.coli DHFR (“eDHFR”).

45. The method of claim 33, wherein the first fusion protein is eDHFR-LexA or R61-LexA.

46. The method of claim 33, wherein the first fusion protein is eDHFR-B42 or R61-B42.

47. The method of claim 33, wherein the reporter gene is Lac Z, ura 3, GFP, β-lactamase, luciferase or an antibody coding region.

48. The method of claim 33, wherein the reporter gene is Lac Z.

49. The method of claim 33, wherein the enzyme that catalyzes bond formation is a transglutaminase.

50. The method of claim 33, wherein the enzyme that catalyzes bond formation is a microbial transglutaminase, a tissue transglutaminase, or Factor XIIIA.

51. A transgenic cell comprising
(a) a dimeric small molecule which comprises a moiety known to bind a receptor domain covalently linked to a first substrate of an enzyme;
(b) nucleotide sequences which upon transcription encode
i) the enzyme,
ii) a first fusion protein comprising the receptor domain, and
ii) a second fusion protein comprising a second substrate of the enzyme; and
(c) a reporter gene wherein expression of the reporter gene is conditioned on the proximity of the first fusion protein to the second fusion protein.

52. The cell of claim 51, wherein the dimeric small molecule has the structure: 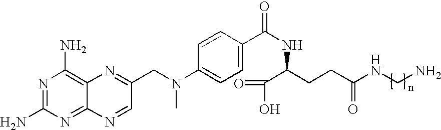
wherein n is an integer from 1 to 20.

53. The cell of claim 52, wherein n is an integer from 2 to 12.

54. The cell of claim 52, wherein n is an integer from 3 to 9.

55. The cell of claim 52, wherein n is 5.

56. The cell of claim 51, wherein the cell is an insect cell, a yeast cell, a bacterial cell, or a mammalian cell.

57. The cell of claim 51, wherein the cell is a yeast cell.

58. The cell of claim 51, wherein the first fusion protein further comprises a DNA binding domain, and the second fusion protein further comprises a transcription activation domain.

59. The cell of claim 51, wherein the first fusion protein further comprises a transcription activation domain, and the second fusion protein further comprises a DNA binding domain.

60. The cell of claim 58 or 59, wherein the DNA-binding domain is LexA, Gal4 or VP16.

61. The cell of claim 58 or 59, wherein the transcription activation domain is B42.

62. The cell of claim 51, wherein the moiety known to bind a receptor domain is a Methotrexate moiety, a dexamethasone moiety, FK506 moiety, an FK506 analog, a teracycline moiety, or a cephem moiety.

63. The cell of claim 51, wherein the known receptor domain is that of dihydrofolate reductase (“DHFR”), glucocorticoid receptor, FKBP12, FKBP mutants, tetracycline repressor, or a penicillin binding protein.

64. The cell of claim 63, wherein the DHFR is the E.coli DHFR (“eDHFR”).

65. The cell of claim 51, wherein the first fusion protein is eDHFR-LexA or R61-LexA.

66. The cell of claim 51, wherein the first fusion protein is eDHFR-B42 or R61-B42.

67. The cell of claim 51, wherein the reporter gene is Lac Z, ura 3, GFP, β-lactamase, luciferase or an antibody coding region.

68. The cell of claim 51, wherein the reporter gene is Lac Z.

69. The cell of claim 51, wherein the first substrate is an amine.

70. The cell of claim 51, wherein the second substrate is an amine.

71. The cell of claim 51, wherein the second substrate is an amino acid sequence containing a lysine.

72. The cell of claim 51, wherein the second substrate is an amino acid sequence containing a glutamine.

73. The cell of claim 51, wherein the second substrate is an amino acid sequence containing-leucine-glycine-glutamine-glycine-.

74. The cell of claim 51, wherein the second substrate is an amino acid sequence containing-leucine-glutamine-glycine-glycine-.

75. The cell of claim 51, wherein the second substrate is an amino acid sequence containing-leucine-leucine-glutamine-glycine-.

76. The cell of claim 51, wherein the second substrate is a modified staphylococcal nuclease (“SNase”) or a modified thioredoxin containing an amino acid sequence containing a glutamine.

77. The cell of claim 51, wherein the enzyme a transglutaminase.

78. The cell of claim 51, wherein the enzyme is a microbial transglutaminase, a tissue transglutaminase, or Factor XIIIA.

79. A kit for detecting bond formation by an enzyme between a first substrate and a second substrate in a cell, comprising
(a) a host cell containing a reporter gene that is expressed only when bound to a DNA-binding domain and when in the proximity of a transcription activation domain;
(b) a first vector containing a promoter that functions in the host cell and a DNA encoding a DNA-binding domain;
(c) a second vector containing a promoter that functions in the host cell and a DNA encoding a transcription activation domain;
(d) a third vector containing a promoter that functions in the host cell;
(e) a dimeric small molecule which comprises a moiety known to bind a receptor domain and a moiety containing the first substrate of the enzyme;
(f) a means for inserting into the first vector or the second vector a DNA encoding a receptor domain in such a manner that the receptor domain and the DNA-binding domain are expressed as a fusion protein;
(g) a means for inserting into the first vector or the second vector a DNA encoding a protein containing the second substrate of the enzyme in such a manner that the protein and the transcription activation domain are expressed as a fusion protein;
(h) a means for inserting into the third vector a DNA encoding the enzyme; and
(h) a means for transfecting the host cell with the first vector, the second vector, and the third vector,
wherein bond formation by the enzyme between the first substrate and the second substrate results in a measurably greater expression of the reporter gene then in the absence of bond formation by the enzyme.

80. A small molecule compound having the structure: 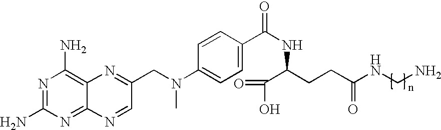
wherein n is an integer from 1 to 20.

81. The compound of claim 80, wherein n is an integer from 2 to 12.

82. The compound of claim 80, wherein n is an integer from 3 to 9.

83. The compound of claim 80, wherein n is 5.
